SEQUENCE LISTING (15 sequences)

SEQ ID NO: 1 - protein, 652 aa, Mus musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDPMDGDTLDFNFDNVLPNQSFPHSVKTTTHSWVSG

SEQ ID NO: 2 - DNA, 2103 bp, Mus musculus
  CDS (7)..(1965)
gtcaccatggccgaggcgccccaggtggtggagaccgacccggacttcgagccgctgccccggcagcgctcctgtacctggccgctgcccaggccggagtttaaccagtccaactcgaccacctccagtccggcgccgtcgggcggcgcggccgccaaccccgacgccgcggcgagcctggcctcggcgtccgctgtcagcaccgactttatgagcaacctgagcctgctggaggagagtgaggacttcgcgcgggcgccaggctgcgtggccgtggcggcggcggctgcggccagcaggggcctgtgcggggacttccagggccccgaggcgggctgcgtgcacccagcgccgccacagcccccaccgaccgggccgctgtcgcagcccccacccgtgcctccctccgctgccgccgccgcggggccactcgcgggacagccgcgcaagaccagctcgtcgcgccgcaacgcgtggggcaacctgtcgtacgccgacctcatcaccaaggccatcgagagctcagccgagaagaggctcaccctgtcgcagatctacgagtggatggtgaagagcgtgccctacttcaaggataagggcgacagcaacagctcggcgggctggaagaattcaattcgccacaatctgtcccttcacagcaagtttattcgagtgcagaatgaaggaactggaaagagttcttggtggatgctcaatccagagggaggcaagagcggaaaatcaccccggagaagagctgcgtccatggacaacaacagtaaatttgctaagagccgagggcgggctgctaagaaaaaagcatctctccagtctgggcaagagggtcctggagacagccctgggtctcagttttctaagtggcctgcgagtcctgggtcccacagcaacgatgactttgataactggagtacatttcgtcctcgaaccagctcaaatgctagtaccatcagtgggagactttctcccatcatgacagagcaggatgacctgggagatggggacgtgcattccctggtgtatccaccctctgctgccaagatggcgtctacgctgcccagtctgtctgaaatcagcaatccagaaaacatggagaaccttctggataatctcaaccttctctcgtccccaacatctttaactgtgtccacccagtcctcgcctggcagcatgatgcagcagacaccatgctattcgtttgcaccgccaaacaccagtctaaattcacccagtccaaactactcaaagtacacatacggccaatccagcatgagccctttgccccagatgcctatgcagacacttcaggacagcaaatcaagttacggaggattgaaccagtataactgtgccccaggactcttgaaagagttgttgacttctgactctcctccccacaatgacattatgtcaccggttgatcccggagtggcccaacccaacagtcgggtcctgggccaaaatgtaatgatgggccctaattcggtcatgccagcgtatggcagccaggcatctcataacaaaatgatgaaccccagctcccacacccaccctggacatgcacagcaaacggcttcggtcaacggccgtaccctgccccatgtggtgaacaccatgcctcacacatctgccatgaaccgcttgacccccgtgaagacacctttacaagtgcctctgtcccaccccatgcagatgagtgccctgggcagctactcctcggtgagcagctgcaatggctatggtaggatgggtgtcctccaccaggagaagctcccaagtgacttggatggcatgtttattgagcgcttggactgtgacatggagtccatcattcggaatgaccccatggatggagataccttggattttaactttgataatgtgttgcccaaccaaagcttcccacacagtgtcaagactacaacacacagctgggtgtcaggctaagagtttagtgagcaggctacatttaaaagtccttcagattgtctgacagcaggaactgaggagcagtccaaagatgcccttcacccctccttatagttttcaagattaaaaaaaaaaaaaaaaaaaaaaaaaaaaaaa

SEQ ID NO: 3 - protein, 652 aa, Mus musculus
MAEAPQVVETDPDFEPLPRQRSCAWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAADMDNNSKFAKSRGRAAKKKASLQSGQEGPGDSPGSQFSKWPASPGSHSNDDFDNWSTFRPRTSANASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDPMDGDTLDFNFDNVLPNQSFPHSVKTTTHSWVSG

SEQ ID NO: 4 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 5 - DNA, 5723 bp, Homo sapiens
  CDS (386)..(2353)
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctcctttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctgtattgtaagcttcccaccgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaa

SEQ ID NO: 6 - protein, 110 aa, Mus musculus
MALWMRFLPLLALLFLWESHPTQAFVKQHLCGSHLVEALYLVCGERGFFYTPMSRREVEDPQVAQLELGGGPGAGDLQTLALEVAQQKRGIVDQCCTSICSLYQLENYCN

SEQ ID NO: 7 - DNA, 460 bp, Mus musculus
  CDS (75)..(407)
gagccctaagtgatccgctacaatcaaaaaccatcagcaagcaggaagcctatcttccaggttattgtttcaacatggccctgtggatgcgcttcctgcccctgctggccctgctcttcctctgggagtcccaccccacccaggcttttgtcaagcagcacctttgtggttcccacctggtggaggctctctacctggtgtgtggggagcgtggcttcttctacacacccatgtcccgccgtgaagtggaggacccacaagtggcacaactggagctgggtggaggcccgggagcaggtgaccttcagaccttggcactggaggtggcccagcagaagcgtggcattgtagatcagtgctgcaccagcatctgctccctctaccagctggagaactactgcaactagacccaccactacccagcctacccctctgcaatgaataaaacctttgaatgagg

SEQ ID NO: 8 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 9 - DNA, 450 bp, Homo sapiens
  CDS (45)..(377)
gctgcatcagaagaggccatcaagcacatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccccacccgccgcctcctgcaccgagagagatggaataaagcccttgaaccagc

SEQ ID NO: 10 - DNA, 22 bp, Artificial
  an artificially synthesized primer sequence
acgttggagagtcatcccgtat

SEQ ID NO: 11 - DNA, 21 bp, Artificial
  an artificially synthesized primer sequence
ctctgtgtggatgcggaagat

SEQ ID NO: 12 - DNA, 25 bp, Artificial
  an artificially synthesized probe sequence
cctgctacatggccacagaccacct

SEQ ID NO: 13 - DNA, 23 bp, Artificial
  an artificially synthesized primer sequence
tcccaggaagatgaagggtttat

SEQ ID NO: 14 - DNA, 22 bp, Artificial
  an artificially synthesized primer sequence
ttccattcgttcgatagcatga

SEQ ID NO: 15 - DNA, 22 bp, Artificial
  an artificially synthesized probe sequence
atgtccccgctcctacaggccc

(19) United States
(12) Patent Application Publication
(10) Pub. No.: US 2007/0238649 A1
(43) Pub. Date: Oct. 11, 2007

(54) Agents for Regulating Adiponectin Receptor Expression

(76) Inventors: Takashi Kadowaki, Tokyo (JP); Toshimasa Yamauchi, Tokyo (JP)

Correspondence Address: Medlen & Carroll, 101 Howard Street, Suite 350, San Francisco, CA, 94105, US

(21) Appl. No.: 10/591,490

(22) PCT Filed: Mar. 4, 2005
(86) PCT No.: PCT/JP05/03744

§ 371 Date: May 10, 2007

Related U.S. Application Data
(60) Provisional application No. 60/549,561, filed on Mar. 4, 2004.

(51) Int. Cl. A61K 38/28; A61P 3/04; A61P 3/08; A61P 7/00; A61P 9/10; C12N 5/06; C12N 5/08; C12Q 1/18; C12Q 1/48

(52) U.S. Cl. 514/3; 435/15; 435/32; 435/375

(57) ABSTRACT

The present inventors revealed by using fasted and refed mice, that AdipoR1/R2 is a regulator of metabolic sensitivity to nutritional conditions and insulin. They showed that mRNA level of AdipoR1/R2 increased by STZ treatment, and that this increase was restored by insulin. The present inventors confirmed in vitro that insulin reduces AdipoR1/R2 mRNAs in myocytes and such. It was also confirmed that in insulin-resistant models, the AdipoR1/R2 expression was downregulated, and that AMP kinase activation by adiponectin was decreased. The present inventors discovered by using insulin signaling pathway inhibitors, that the downregulation of adiponectin receptors by insulin was mediated by the PI3-kinase/Foxo1 pathway.

### SUMMARY

Adiponectin/Acrp30 is a hormone secreted by adipocytes that acts as an antidiabetic and anti-atherogenic adipokine. We reported that AdipoR1/R2 serve as receptors for adiponectin and mediate increased fatty-acid oxidation and glucose uptake by adiponectin. In this study, we studied expression levels and roles of AdipoR1/R2 in several physiological and pathophysiological states such as fasting/refeeding, obesity and insulin resistance. Here we show that the expressions of AdipoR1/R2 in insulin target organs such as skeletal muscle and liver are significantly increased in fasted mice and decreased in refed mice. The expression of AdipoR1/R2 appears to be inversely correlated with plasma insulin levels in vivo. Indeed, STZ increased and insulin reduced the expression of AdipoR1/R2 in vivo. Interestingly, incubation of hepatocytes or myocytes with insulin reduced the expression of AdipoR1/R2 via PI3-kinase/Foxo1 dependent pathway in vitro. Moreover, the expressions of AdipoR1/R2 of ob/ob mice are significantly decreased in skeletal muscle and adipose tissue, which were also correlated with decreased adiponectin binding to membrane fractions of skeletal muscle and decreased AMP kinase activation by adiponectin. This subsequent decrease in adiponectin effects called “adiponectin resistance” in turn may play a role in worsing insulin resistance in ob/ob mice. In conclusion, the expression of AdipoR1/R2 appears to be regulated by several physiological and pathophysiological states such as fasting/refeeding and hyperinsulinemia via insulin/PI3-kinase/Foxo1 pathway, and correlated with adiponectin sensitivity.

Introduction

Adiponectin/Acrp30 (1-4) is a hormone secreted by adipocytes that acts as an antidiabetic (5-12) and anti-atherogenic (8,12,13) adipokine. This insulin sensitizing effect of adiponectin appears to be mediated by an increase in fatty acid oxidation via activation of the 5′-AMP-activated protein kinase (AMPK) (10,11) and peroxisome proliferator-activated receptor (PPAR)α (5,6,12). Very recently, we have reported the cloning of complementary DNAs encoding adiponectin receptors (AdipoR) 1 and 2 by expression cloning (14). AdipoR1 is abundantly expressed in skeletal muscle, whereas AdipoR2 is predominantly expressed in the liver. AdipoR1 and R2 are predicted to contain seven transmembrane domains (14), but to be structurally and functionally distinct from G-protein coupled receptors (15-17). AdipoR1 and R2 serve as receptors for globular and full-length adiponectin, and mediate increased AMPK (10,11), PPARα ligands activities (12), and the fatty-acid oxidation and glucose uptake by adiponectin (14).

It has not been shown previously whether the expressions of AdipoR1 and R2 are altered in physiological and pathophysiological states or whether they are correlated with adiponectin sensitivity. To address these questions, we first studied the expressions of AdipoR1 and R2 during fasting and refeeding, and showed that the expression of AdipoR1/R2 is altered dramatically during fasting and refeeding of mice, closely paralleling the regulation of plasma insulin levels. The expression of AdipoR1/R2 appears to be inversely correlated with plasma insulin levels in vivo. These data identify AdipoR1/R2 as an important regulator of metabolism sensitive to nutritional status and insulin.

In the current studies, we next have analyzed the effect of insulin on the levels of the AdipoR1/R2 mRNAs in livers, skeletal muscles and adipose tissues of living mice. For this purpose, we treated mice with streptozotocin (STZ), which destroys the β-cells of the pancreas and leads to an acute insulin deficiency (18). We show that this treatment leads to a profound increase in AdipoR1/R2 mRNA, and this is restored by administration of insulin. These data support an in vivo role for insulin in specifically repressing transcription driven by the AdipoR1/R2 promoter.

Moreover, we show here that insulin reduces AdipoR1/R2 mRNA in myocytes and hepatocytes. We next addresses the mechanism of insulin signalling for reduction of AdipoR1/R2 gene expression in myocytes. Interestingly, insulin incubation with insulin reduced the expression levels of AdipoR1/R2 via PI3-kinase/Foxo1 pathway (19) in vitro.

We also studied these expressions in models of altered insulin sensitivity such as an obesity-linked insulin resistance model. The expressions of AdipoR1/R2 of ob/ob mice are significantly decreased in muscle and adipose tissue, which were also correlated with decreased adiponectin binding to membrane fractions of these tissues and decreased AMP kinase activation by adiponectin. The subsequent decrease in adiponectin effects called “adiponectin resistance” in turn may play a role in worsing insulin resistance in ob/ob mice.

In conclusion, the expression of AdipoR1/R2 appears to be regulated by several physiological and pathophysiological states such as fasting/refeeding and hyperinsulinemia, and correlated with adiponectin sensitivity. Our data also suggested that not only agonism of AdipoR1/R2 but also the strategies to increase AdipoR1/R2 may be a logical approach to provide a novel treatment modality for insulin resistance and type 2 diabetes.

### Experimental Procedures

Chemicals All materials were from the sources given in the References (6, 10, 14 and 20).

Animals Fifteen-week-old ob/ob mice and their wild-type C57BL/6 mice were obtained from CharlesRiver Breeding Laboratories (Wilmington, Mass.). Male mice were housed in colony cages, maintained on a 12-h light/12-h dark cycle. Our high-fat diet contains oil 1152 g (from Benibana, Japan; safflower oil [high-oleic type] contained 46% oleic acid (18:1n-9) and 45% linoleic acid (18:2n-6) from total fatty acids), casein 1191.6 g (Oriental Yeast, Tokyo, Japan, No.19), sucrose 633.6 g (Oriental Yeast, No. 13), vitamin mix 50.4 g (Oriental Yeast, No. 20 (AIN76), mineral mix 352.8 g (Oriental Yeast, No.25 (AIN76), cellulose powder 201.6 g (Oriental Yeast, No.19), DL-methionine 18 g (Wako Pure Chemicals, Osaka, Japan), water 360 ml; total 3600 g (20). Plasma insulin was measured by an insulin immunoassay (Shibayagi, Gunma, Japan). The animal care and procedures were approved by the Animal Care Committee of the University of Tokyo.

STZ Treatments Diabetes was induced by a double intraperitoneal injection of 0.2-0.3 ml of 50 mM sodium citrate solution (pH 4.5) containing STZ (200 mg/kg body weight). Control (non-STZ) mice were injected with 50 mM sodium citrate solution (pH 4.5). Three days after injection, plasma glucose levels were checked and diabetes was confirmed (glucose level >250 mg/dl). A combination of human regular insulin (1 units/kg; Eli Lilly, Indianapolis, Ind.), each given in 0.2 ml of saline was administered to the STZ+ insulin group. The mice in the STZ+ vehicle and non-STZ groups received 0.2 ml of saline injected both intraperitoneally. After injection of INS or saline, the animals were fasted for 6 h and then hindlim's skeletal muscles were removed.

Fasting and Refeeding In the refeeding experiment, food was reintroduced after 48 h of fasting. Animals were killed after 6 h from refeeding for the isolation of tissues.

Mice Primary Hepatocytes Hepatocytes were isolated from fasting 8-week old male C57BL/6 mice by the collagenase perfusion method (21). Animals were anesthetized with pentobarbital, and each liver was perfused in situ via the portal vein with 10 ml of a perfusion medium (0.8% NaCl, 5 mM KCl, 0.5 mM NaH₂PO₄, 0.42 mM Na₂HPO₄, 10 mM Hepes, 0.5 mM EGTA, 4.1 mM NaHCO₃, 5 mM glucose). The medium was warmed to 37° C. and infused at a rate of [approx] 6 ml/min. The liver was then perfused with a digestion medium (0.8% NaCl, 5 mM KCl, 5 mM CaCl2, 0.5 mM NaH₂PO₄, 0.42 mM Na₂HPO₄, 10 mM Hepes, 4.1 mM NaHCO₃, 0.4 mg/ml collagenase, 0.28 mg/ml trypsin inhibitor). for [approx] 5 min at a flow rate of [approx] 6 ml/min. The liver was removed, the hepatic capsule was stripped, and the dissociated cells were dispersed by shaking, followed by filtration at 4° C. through gauze into an equal volume of ice-cold Williams medium E (GIBCO/BRL, Grand Island, N.Y.). The cells were pelleted and washed forth at 4° C. with the same medium. Cell viability, as measured by trypan blue exclusion, was always greater than 85%. Aliquots of 0.8×10⁶ cells were plated onto collagen I-coated 12-well dishes (Iwaki, Chiba, Japan) in Williams medium E supplemented with 5% (vol/vol) fetal calf serum, 10 nM dexamethazone, 1 nM insulin, 100 units/ml of penicillin, and 100 μg/ml of streptomycin. After incubation at 37° C. in 5% CO₂ for 5 h, the cells were washed twice with PBS and incubated with Williams medium E supplemented with 1% bovine serum albumin for 6 h. Then, the cells were switched to Williams medium E supplemented with 1% bovine serum albumin and 100 nM insulin and harvested 6 h after insulin addition for isolation of total RNA.

Studies with C2C12 and 3T3L1 cells Induction of myogenic differentiation was carried out according to a method described previously (10). In brief, mouse C2C12 myoblasts were grown in Dulbecco's modified high-glucose Eagle's medium (DMEMH; 90%)/10%(v/v) FBS. When the cells were 80% confluent, myoblasts were induced to differentiate into myotubes by replacing the medium with a low-serum differentiation medium [98% DMEMH/2.5% (v/v) horse serum], which was changed daily. By day 5, the cells had differentiated into multinucleated, contracting myotubes.

Inhibition of PI3-kinase pathways C2C12 cells were incubated with DMEMH supplemented with 1% bovine serum albumin for 12 h. Inhibitors(10 μM LY294002 or 10 μM PD98059) were supplied to cells 30 min before insulin addition. Cells were harvested 6 h after insulin addition for isolation of total RNA.

Adenovirus-mediated gene transfer of Foxo1 The murine cDNAs encoding wild-type (WT), T24A/S253D/S316A (ADA), and Δ256 mutant Foxo1, which includes the entire forkhead domain and lacks the transactivation domain, were used (22). C2C12 cells were transfected with adenovirus containing cDNAs described above, as previously described (10), with some modifications. Induction of differentiation was carried out according to methods described above. Five days later, the cells were treated with the indicated concentrations of insulin, and then subjected to analysis.

Quantitative analysis of AdipoR1 and R2 transcripts by Northern blot and Real-time PCR Total RNA was prepared from cells or tissues with TRIzol (GIBCO/BRL) according to the manufacturer's instructions. For quantification of AdipoRs mRNAs, we employed the real-time PCR method (14). The primer sets and the probes for mAdipoR1,2 were as follows; the forward primer for mAdipoR1 was acgttggagagtcatcccgtat, the reverse primer, ctctgtgtggatgcggaagat and the probe, cctgctacatggccacagaccacct; the forward primer for mAdipoR2 was tcccaggaagatgaagggtttat, the reverse primer, ttccattcgttcgatagcatga and the probe, atgtccccgctcctacaggccc. The relative amount of each AdipoR transcript was normalized to the amount of actin transcript in the same cDNA (14).

Generation of recombinant adiponectin, adiponectin tolerance test and Western blot analysis Bacterially expressed murine full-length adiponectin (Ad) was generated as previously described (10, 14). Fifteen-old male ob/ob mice or age-matched C57BL/6 mice were anesthetized with pentvarvital and then injected with recombinant adiponectin (50 μg/10 g body weight) via vena cava inferior. Skeletal muscles were isolated 5 min after adiponectin injection. Phosphorylation and amount of AMPK in keletal muscles were determined (10, 14).

Binding Assay Synthetic adiponectin was ¹²⁵I-labeled at Tyr by IODO-beads (Pierce) in the presence of Na¹²⁵I (2,000 Ci/mmol, Amersham Pharmacia Biotech) according to the manufacturer's protocol. Membrane fractions of muscles were incubated at 4° C. for 1 hr with binding buffer (ice-cold phosphate-buffered saline/0.1% bovine serum albumin) containing designated concentrations of [¹²⁵I]adiponectin (5,000 cpm/ng protein) plus unlabeled competitors. The binding equilibrium was found to be established when the binding assay was conducted at 4° C. after 1 h. The cells were then washed three times with ice-cold phosphate-buffered saline, lysed in 0.1 N NaOH/0.1% SDS, and the cell-bound radioactivity was determined using a γ-counter (14). Nonspecific binding was determined using a 200-fold excess of unlabeled adiponectin. Specific binding was calculated by subtracting nonspecific binding from the total binding. The values presented in the “Results” represent an average of triplicate determinations of 3-10 experiments.

### Results

Fasting in Creased AdipoR1/R2 Gene Expressions, Whereas Refeeding Decreased Them

To clarify whether expressions of AdipoR1 and/or AdipoR2 would be regulated under physiological and/or pathophysiological states, we first examined the expression levels of AdipoR1 and AdipoR2 in insulin and also adiponectin target organs such as skeletal muscle and liver from the mice under ad libitum-fed, fasted and refed conditions. At the start of the night cycle (9:00 p.m.), animals were either provided with food or deprived of food for 48 h. A third group of mice was refed for 6 h after the 48-h fast. As can be seen in FIGS. 1A and 1B, AdipoR1 and AdipoR2 mRNA expression in the liver increased dramatically after the 48-h fast, and refeeding rapidly restored these to a level equal to the original fed state. We have found that similar regulation of AdipoR2 mRNA by fasting and refeeding is seen in the skeletal muscle (FIGS. 1C and 1D).

Plasma Insulin Levels Appear to be Inversely Correlated with Expression Levels of AdipoR1/R2 Gene

The particular hormones or nutrients responsible for this response to fasting and feeding are of considerable interest. Since insulin is the classic hormone of the fed state, we next measured the plasma insulin levels under ad libitum-fed, fasted and refed states. As expected, fasting decreased the plasma insulin levels (FIG. 1E), and refeeding increased them in a parallel fashion with the plasma glucose levels (FIG. 1F). These data suggested that plasma insulin levels appear to be inversely correlated with expression levels of AdipoR1/R2 gene, and raised the possibility that insulin may control AdipoR1/R2 expression.

STZ Treatment Increased AdipoR1/R2 Expressions, Whereas Insulin Decreased Them

To address this issue, we next studied the effects of insulin on AdipoR1/R2 expression in mice treated with STZ. To eliminate the effects of insulin on food intake, we withdrew food from all of the mice and killed the animals 6 h later. STZ treatment abolished plasma insulin (FIG. 2A) and at the same time caused a significant marked increase in plasma glucose (FIG. 2B, lanes 1, 2). The animals treated with STZ plus insulin showed a reduced plasma glucose levels when compared with the animals treated with STZ alone (FIG. 2B, lanes 2, 3).

We next measured the amounts of mRNA for AdipoR1 and R2 in skeletal muscle and liver extracts. The results revealed an 103% and 38% increase in AdipoR1 and Adipo R2 mRNA, respectively, in skeletal muscle of the STZ-treated mice that was restored nearly completely by insulin treatment (FIGS. 2C and 2D). These data further supported that insulin would reduce AdipoR1/R2 mRNA levels. STZ treatment had little effect on AdipoR1/R2 mRNA levels in the liver, whereas insulin decreased the AdipoR1/R2 mRNA levels in the liver of STZ-treated mice (data not shown). These data may be explained by the observations that the inflammation may decrease AdipoR1/R2 mRNA levels (Tsuchida, Yamauchi, Kadowaki, manuscript in preparation) and that STZ treatment may induce inflammation in the liver (23).

Insulin Decreased AdipoR1/R2 mRNA Levels In Vitro

To determine whether insulin has a direct effect on AdipoR1/R2 expression in hepatocytes, we isolated hepatocytes from livers of the C57BL/6 mice and incubated them for 5 h in medium containing FBS, insulin and dexamethasone, a mixture that preserves the differentiated state of freshly isolated hepatocytes (21). We then starved the cells for 6 h in the same medium without FBS, insulin and dexamethasone, and then incubated them for a further 6 h with or without 100 nM insulin. The hepatocytes were then harvested, and we measured the amounts of total AdipoR1 and R2 mRNA. Insulin produced an decrease in the total amount of AdipoR1/R2 mRNA, (FIGS. 3A and 3B). To further address the possibility that insulin may reduce AdipoR1/R2 mRNA levels, we next studied whether insulin would reduce AdipoR1/R2 mRNA levels in C2C12 myocytes in vitro. Insulin treatment at 10 nM indeed decreased AdipoR1/R2 expression after 6 h (FIGS. 3C and 3D).

Insulin Reduction of AdipoR1/R2 mRNA is Suppressed by Inhibitors of Phosphoinositide 3-Kinase (PI 3-Kinase)

The level of AdipoR1/R2 mRNA in C2C12 myocytes was investigated 6 h after the addition of insulin to myocytes incubated with various inhibitors of insulin signalling pathways (FIGS. 3C and 3D). The inhibitor of PI 3-kinase, LY 294002, abolished the decreaed effect of insulin. In contrast, the response to insulin was essentially unaffected by PD 98059; an inhibitor of mitogen-activated protein kinase activation. The inhibitor effects shown in FIGS. 3C and 3D were maximal effects for all of the drugs tested, a point that was verified by dose-response experiments (results not shown).

Foxo1 Increased AdiporR1/R2 mRNA Levels and Blocked Insulin-Induced Reduction of Them

Insulin-dependent repression of genes, which harbours a cis-acting element capable of binding Foxo1 in its promoter, might depend, at least in part, on PI3-kinase/Akt-mediated phosphorylation and inactivation of this forkhead transactivator (22). Expression of constitutively active form of Foxo1 (Foxo1-ADA) caused a marked increase in AdipoR1/R2 mRNA levels. Insulin could not reverse the effect of Foxo1 on AdipoR1/R2 mRNA levels (FIGS. 3E and 3F). These data suggested that Foxo1 increased expression levels of AdipoR1/R2, and that insulin repressed AdipoR1/R2 mRNA expressions via inactivation of Foxo1.

AdipoR1/R2 Expression Levels Were Downregulated in Ob/Ob Mice

We studied the expression of AdipoR1 and R2 in insulin sensitive tissues such as liver, skeletal muscle such as the soleus muscle and extensor digitorum longus (EDL), and adipose tissues such as WAT and BAT in control C57BL/6 mice and insulin resistant ob/ob mice. As shown in FIGS. 4A and 4B, in control C57BL/6 mice, AdipoR1 was most abundantly expressed in skeletal muscle and AdipoR2 was most abundantly expressed in the liver, as we have previously reported (14). Interestingly, both AdipoR1 and R2 were more abundantly expressed in the EDL than in the soleus muscle. Moreover, both WAT and BAT expressed AdipoR1 and R2.

The expressions of both AdipoR1 and R2 were significantly decreased in most of the insulin sensitive tissues examined but not liver in ob/ob mice as compared with control C57BL/6 mice (FIGS. 4A and 4B). The expressions of AdipoR1 in the liver, soleus, EDL, WAT, and BAT from ob/ob mice were 79.4%, 54.5%, 50.5%, 31.9%, and 28.1% of those from the wild-type mice, respectively (FIG. 4A). The expressions of AdipoR2 in the liver, soleus, EDL, WAT, and BAT from ob/ob mice were 63.3%, 51.4%, 59.5%, 7.9%, and 29.4% of those from the wild-type mice, respectively (FIG. 4B).

Decreased Expression Levels of AdipoR1/R2 in Ob/Ob Mice Apear to be Correlated with Decreased “Adiponectin Sensitivity”

To clarify whether expression levels of AdipoR1/R2 are associated with adiponectin effects in each tissues, we measured adiponectin binding to the membrane fractions of skeletal muscle and AMPK activation by adiponectin. Interestingly, adiponectin had a higher binding to the membrane fractions of skeletal muscle of wild-type mice than ob/ob mice (FIGS. 5A and 5B). Moreover, adiponectin were able to activate AMPK in skeletal muscle of wild-type mice whereas adiponectin were unable to activate AMPK (FIGS. 5C and 5D). These data suggested that expression levels of AdioR1/R2 might regulate adiponectin binding and AMPK activation by adiponectin called “adiponectin sensitivity”

### Discussion

We have recently cloned the adiponectin receptors AdipoR1 and AdipoR2 which appear to mediate the insulin sensitizing actions of adiponectin (14). In the present study, we examined the expression levels of AdipoR1 and R2 to explore the potential role of altered expression of adiponectin receptors in the regulation of insulin and/or adiponectin sensitivity. Interestingly, we showed for the first time in this study that fasting increased the expression levels of AdipoR1 and R2, whereas refeeding decreased them.

The last 20 years have seen a vast increase in our knowledge of how nutrients and hormones controlled by diet can influence gene expression. The classic hormones of the fasted state, catecholamines and glucagon, function by raising cAMP levels, which can result in the activation of the transcription factor cAMP response element-binding protein through protein kinase A (24). This factor then functions in a variety of fasting-sensitive genes through a canonical cAMP response element-binding protein binding site. Fasting also leads to an elevation of glucocorticoids, which control gene expression mainly if not exclusively through the glucocorticoid receptor and its well-defined cognate sequence (25). However, cAMP nor glucocorticoids did not have appeared marked effects on expression levels of AdipoR1/R2 (Tsuchida, Yamauchi and Kadowaki, unpublished data).

We next examined whether insulin might affect the expression levels of AdipoR1/R2. Interestingly, streptozotocin (STZ) treatment diminished plasma insulin and at the same time caused a marked increase in AdipoR1/R2 mRNA levels and replenishment of insulin into STZ-treated mice reduced them to those observed in mice without STZ treatment, indicating that insulin reversed the effect of STZ on AdipoR1/R2 mRNA levels. It decreased greatly their expressions at the mRNA level and does so at doses of insulin consistent with a function through the insulin receptor. Of course, it is entirely possible that other hormones or nutrients in addition to insulin participate in the regulation of AdipoR1/R2 in this in vivo context.

For a factor to be described as an insulin response factor, key criteria must be satisfied. The target DNA sequence to which the gene control factor binds must be characterized. Most, if not all, of the known actions of insulin can be attributed to the receptor-mediated tyrosine-phosphorylation of IRS-1 and IRS-2, which in turn triggers kinase cascades involving the phosphatidylinositol 3-kinase (PI 3-kinase)/Akt pathway and the MAP kinase pathway (26-28). With respect to AdipoR1/R2 gene expression, insulin signalling via several, possibly redundant, pathways remains an intriguing possibility. The insulin effect on AdipoR1/R2 gene expression was virtually unaffected by the MEK inhibitor, PD 98059, and thus does not appear to require activation of the mitogen-activated protein kinase cascade. By contrast, results with LY 294002 clearly implicated the PI 3-kinase signalling pathway. These results of experiments using a inhibitor of PI 3-kinase, lead us to propose that induction of AdipoR1/R2 gene expression is most likely mediated via the PI 3-kinase branch of the insulin signalling pathways.

The PI 3-kinase signalling pathway is thought to be involved in the insulin regulation of several genes, notably the gene for the insulin-like growth-factor binding protein (IGF-BP) 1 (29). Recently, Akt, which is the downstream effector of PI3-kinase, was shown to phosphorylate several transcriptional activators of the forkhead family, including Foxo1, in vitro and in intact cells. The phosphorylation of specific residues in these factors inhibited their ability to activate transcription of target genes as a result of the sequestration of the phosphorylated factors in the cytoplasm (30-32). Thus, the insulin-dependent repression of the IGF-BP1 gene, which harbours a cis-acting element capable of binding Foxo1 in its promoter, is thought to depend, at least in part, on Akt-mediated phosphorylation and inactivation of this forkhead transactivator (29).

The DNA-binding characteristics are noteworthy because several (but not all) identified insulin response elements appear to be recognized by Foxo1. Visual and computer-assisted examination reveal many potential responsible sites, a sequence as an insulin response element in the AdipoR1/R2 promoter. Thus we next examined whether Foxo1 would regulate the AdipoR1/R2 expressions. Interestingly, Foxo1 increased the AdipoR1/R2 expressions and insulin was unable to reduce them, suggesting that Foxo1 increased the AdipoR1/R2 expressions and insulin reduced them via downregulation of Foxo1 activity it is clear that AdipoR1/R2 is functioning as an insulin response factor, at least in muscle cells. Of course, further determination of the relative importance of insulin in the regulation of AdipoR1, and AdipoR2 genes must await loss-of-function experiments involving genetic ablation of insulin in various tissues. To date, there is no data indicating that ectopic expression of other factors can regulate AdioR1/R2 expression. Further studies will be required to clarify this point.

Finally, we found that the expressions of AdipoR1 and AdipoR2 were downregulated in ob/ob mice, a model of insulin resistance linked to obesity. We have previously shown that plasma adiponectin levels were decreased in ob/ob mice (12) and that these alterations in plasma adiponectin levels may play causal roles in the regulation of insulin sensitivity. These in combination raise the interesting possibility that altered expressions of AdipoR1/R2 in addition to plasma adiponectin levels may play a causal role in the regulation of insulin sensitivity. This is consistent with our previous observation that decreased expressions of AdipoR1 or AdipoR2 by their respective siRNA were associated with the reduced insulin sensitizing effects of adiponectin (14). We provided the first evidence that the decreased expressions of AdipoR1 or AdipoR2 in these mouse models are associated with reductions of the insulin sensitizing effects of adiponectin in vivo. These data also suggest the possibility that plasma insulin concentrations regulate AdipoR1/R2 expressions, although it is also possible that ambient adiponectin concentrations positively regulate the expression levels of AdioR1 and AdipoR2. This is the first demonstration, to the best of our knowledge, that the expressions of AdipoR1/R2 in muscle and adipocytes appear to be inversely correlated with plasma insulin levels.

Hence, chronic overfeeding and its attending elevation of insulin would be expected to result in decreased expression of AdipoR1/R2. This is likely to affect both adiponectin, and more insulin sensitivity. The current results in the muscles of intact mice with STZ-induced diabetes, together with those in C2C12 myocytes, indicate that insulin reduces AdipoR1/R2 gene transcription. The decrease in AdipoR1/R2 mRNA leads to a decrease in the adiponectin binding, and this in turn leads to a decrease in adiponectin effects called “adiponectin resistance”. These findings appear to explain, at least in part, the long-appreciated ability of insulin to induce insulin resistance.

In conclusion, the expression of AdipoR1/R2 appears to be regulated by several physiological and pathophysiological states such as fasting/refeeding and hyperinsulinemia, and correlated with adiponectin sensitivity. Our data also suggested that not only agonism of AdipoR1/R2 but also the strategies to increase AdipoR1/R2 may be a logical approach to provide a novel treatment modality for insulin resistance and type 2 diabetes.

Acknowledgements

We thank Dr. H. Waki, S. Uchida, and S. Kita for their helpful suggestions. We are grateful to A. Itoh, A. Okano, L. Yao and K. Kirii for their excellent technical assistance. This work was supported by the Program for Promotion of Fundamental Studies in Health Sciences of the Organization for Pharmaceutical Safety and Research of Japan, a grant from the Human Science Foundation (to T.K.), a Grant-in-Aid for the Development of Innovative Technology from the Ministry of Education, Culture, Sports, Science and Technology (to T.K.), and a Grant-in Aid for Creative Scientific Research 10NP0201 from the Japan Society for the Promotion of Science (to T.K.), and by Health Science Research Grants (Research on Human Genome and Gene Therapy) from the Ministry of Health, Labour and Welfare (to T.K.).

### REFERENCES

1. Scherer, P. E., Williams, S., Fogliano, M., Baldini, G., and Lodish, H. F. (1995) J. Biol. Chem. 270, 26746-26749
2. Hu, E., Liang, P., and Spiegelman, B. M. (1996) J. Biol. Chem. 271, 10697-10703
3. Maeda, K., Okubo, K., Shimomura, I., Funahashi, T., Matsuzawa, Y., and Matsubara, K. (1996) Biochem. Biophys. Res. Commun. 221, 286-296
4. Nakano, Y., Tobe, T., Choi-Miura, N. H., Mazda, T., and Tomita, M. (1996) J. Biochem. (Tokyo) 120, 802-812
5. Fruebis, J., Tsao, T. S., Javorschi, S., Ebbets-Reed, D., Erickson, M. R., Yen, F. T., Bihain, B. E., and Lodish, H. F. (2001) Proc. Natl. Acad. Sci. USA. 98, 2005-2010
6. Yamauchi, T., Kamon, J., Waki, H., Terauchi, Y., Kubota, N., Hara, K., Mori, Y., Ide, T., Murakami, K., Tsuboyama-Kasaoka, N., Ezaki, O., Akanuma, Y., Gavrilova, O., Vinson, C., Reitman, M. L., Kagechika, H., Shudo, K., Yoda, M., Nakano, Y., Tobe, K., Nagai, R., Kimura, S., Tomita, M., Froguel, P., and Kadowaki, T. (2001) Nat. Med. 7, 941-946
7. Berg, A. H., Combs, T. P., Du, X., Brownlee, M., and Scherer, P. E. (2001) Nat. Med. 7, 947-953
8. Kubota, N., Terauchi, Y., Yamauchi, T., Kubota, T., Moroi, M., Matsui, J., Eto, K., Yamashita, T., Kamon, J., Satoh, H., Yano, W., Froguel, P., Nagai, R., Kimura, S., Kadowaki, T., and Noda, T. (2002) J. Biol. Chem. 277, 25863-25866
9. Maeda, N., Shimomura, I., Kishida, K., Nishizawa, H., Matsuda, M., Nagaretani, H., Furuyama, N., Kondo, H., Takahashi, M., Arita, Y., Komuro, R., Ouchi, N., Kihara, S., Tochino, Y., Okutomi, K., Horie, M., Takeda, S., Aoyama, T., Funahashi, T., and Matsuzawa, Y. (2002) Nat. Med. 8,731-737
10. Yamauchi, T., Kamon, J., Minokoshi, Y., Ito, Y., Waki, H., Uchida, S., Yamashita, S., Noda, M., Kita, S., Ueki, K., Eto, K., Akanuma, Y., Froguel, P., Foufelle, F., Ferre, P., Carling, D., Kimura, S., Nagai, R., Kahn, B.B., and Kadowaki, T. (2002) Nat. Med. 8, 1288-1295
11. Tomas, E., Tsao, T. S., Saha, A. K., Murrey, H. E., Zhang, Cc. C., Itani, S. I., Lodish, H. F., and Ruderman, N. B. (2002) Proc. Natl. Acad. Sci. USA. 99, 16309-16313
12. Yamauchi, T., Kamon, J., Waki, H., Imai, Y., Shimozawa, N., Hioki, K., Uchida, S., Ito, Y., Takakuwa, K., Matsui, J., Takata, M., Eto, K., Terauchi, Y., Komeda, K., Tsunoda, M., Murakami, K., Ohnishi, Y., Naitoh, T., Yamamura, K., Ueyama, Y., Froguel, P., Kimura, S., Nagai, R., and Kadowaki, T. (2003) J. Biol. Chem. 278, 2461-2468
13. Ouchi, N., Kihara, S., Arita, Y., Nishida, M., Matsuyama, A., Okamoto, Y., Ishigami, M., Kuriyama, H., Kishida, K., Nishizawa, H., Hotta, K., Muraguchi, M., Ohmoto, Y., Yamashita, S., Funahashi, T., and Matsuzawa, Y. (2001) Circulation 103, 1057-1063
14. Yamauchi, T., Kamon, J., Ito, Y., Tsuchida, A., Yokomizo, T., Kita, S., Sugiyama, T., Miyagishi, M., Hara, K., Tsunoda, M., Murakami, K., Ohteki, T., Uchida, S., Takekawa, S., Waki, H., Tsuno, N. H., Shibata, Y., Terauchi, Y., Froguel, P., Tobe, K., Koyasu, S., Taira, K., Kitamura, T., Shimizu, T., Nagai, R., and Kadowaki, T. (2003) Nature 423, 762-769
15. Wess, J. (1997) FASEB. J. 11, 346-354
16. Yokomizo, T., Izumi, T., Chang, K., Takuwa, Y., and Shimizu, T. (1997) Nature 387, 620-624
17. Scheer, A., Fanelli, F., Costa, T., De Benedetti, P. G., and Cotecchia, S. (1996) EMBO. J. 15, 3566-3578
18. Rakieten, N., Rakieten, M I., and Nadkarni, M V. (1963) Cancer Chemother. Rep. 29, 91-98
19. Nakae, J., Park, B.-C., and Accili, D. (1999) J. BIol. Chem. 274, 15982-15985
20. Yamauchi, T., Waki, H., Kamon, J., Murakami, K., Motojima, K., Komeda, K., Miki, H., Kubota, N., Terauchi, Y., Tsuchida, A., Tsuboyama-Kasaoka, N., Yamauchi, N., Ide, T., Hori, W., Kato, S., Fukayama, M., Akanuma, Y., Ezaki, O., Itai, A., Nagai, R., Kimura, S., Tobe, K., Kagechika, H., Shudo, K., and Kadowaki, T. (2001) J. Clin. Invest. 108, 1001-1013
21. Seglen, P O. (1976) Methods Cell. Biol. 13, 29-83
22. Nakae, J., Kitamura, T., Siliver, D L., and Accili, D. (2001) J. Clin. Invest. 108, 1359-1367
23. Levine, B S., Henry, M C., Port, C D., and Rosen, E. (1980) Drug. Chem. Toxicol. 3, 201-212
24. Herzig, S., Long, F., Jhala, U S., Hedrick, S., Quinn, R., Bauer, A., Rudolph, D., Schutz, G., Yoon, C., Puigserver, P., Spiegelman, B., and Montminy, M. Nature (2001) 13, 179-83.
25. Friedman, J E., Sun, Y., Ishizuka, T., Farrell, C J., McCormack, S E., Herron, L M., Hakimi, P., Lechner, P., and Yun, J S. J. Biol. Chem. (1997) 272, 31475-31481
26. Shepherd, P. R., Withers, D. J., and Siddle, K. Biochem. J. (1998) 333, 471-490
27. Kadowaki, T. J. Clin. Invest. (2000) 106, 459-465.
28. Virkamaki, A., Ueki, K., and Kahn, C R. J. Clin. Invest. (1999) 103, 931-943
29. Guo, S., Rena, G., Cichy, S., He, X., Cohen, P., and Untertman, T. J. Biol. Chem. (1999) 274, 17184-17192
30. Guo, S., Rena, G., Cichy, S., He, X., Cohen, P., and Unterman, T. J. Biol. Chem. (1999) 274, 17184-17192
31. Biggs, W. H., Meisenhelder, J., Hunter, T., Cavenee, W. K., and Arden, K. C. Proc. Natl. Acad. Sci. U.S.A. (1999) 96, 7421-7426
32. Takaishi, H., Konishi, H., Matsuzaki, H., Ono, Y., Shirai, Y., Saito, N., Kitamura, T., Ogawa, W., Kasuga, M., Kikkawa, U., and Nishizuka, Y. Proc. Natl. Acad Sci. U.S.A. (1999) 96, 11836-11841
33. Brunet, A., Bonni, A., Zigrnond, M. J., Lin, M. Z., Juo, P., Hu, L. S., Anderson, M. J., Arden, K. C., Blenis, J., and Greenberg, M. E. Cell (1999) 96, 857-868

### FIGURES LEGENDS

FIG. 1. Nutritional regulation of AdipoR1/R2 gene expression. (A) mRNA expression levels of AdipoR1 (A, C) and AdipoR2 (B, D) in liver (A, B) and skeletal muscle (C, D), blood glucose (E) and plasma insulin (F) of mice were compared. A fed control (ad lib) was allowed free access to food (lane 1). A fasted group (Fasted) were denied access to food for 48 hours (lane 2). The refed group (Refed) was allowed free access to food for 6 hours after 48 hours of fasting (lane 3). Total RNA was prepared from these tissues with TRIzol. For quantification of AdipoRs mRNAs, we employed the real-time PCR method. The primer sets and the probes for mAdipoRs were as indicated in Research Design and Methods. The relative amount of each AdipoR transcript was normalized to the amount of β-actin transcript in the same cDNA. Each bar represents the mean±s.e. (n=3). (*, P<0.05; **, P<0.01; compared with fasted mice).

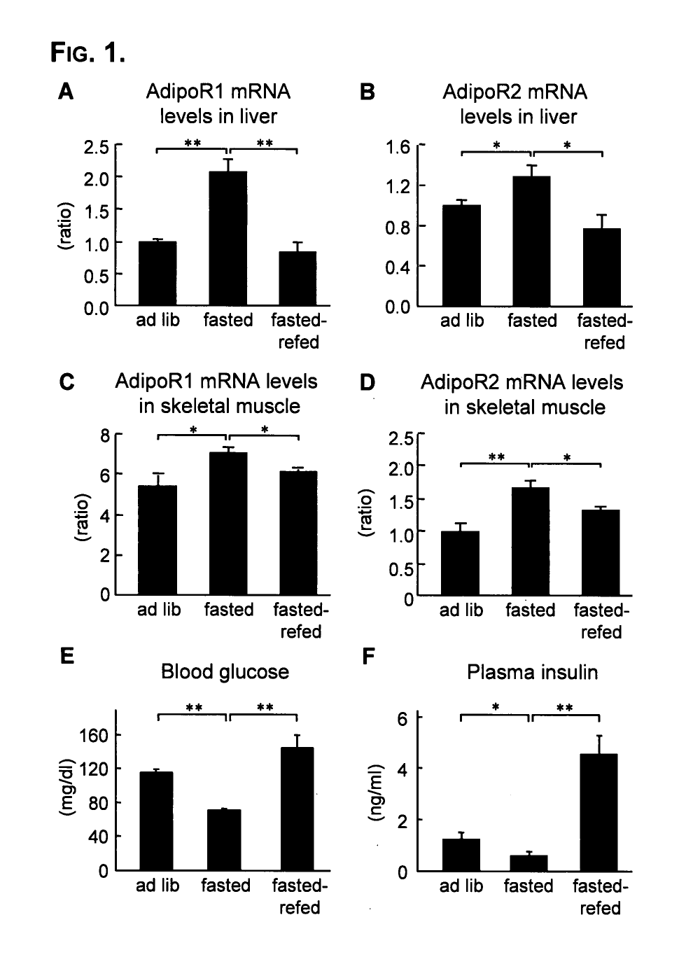

FIG. 2. Plasma insulin (A), blood glucose (B), amounts of AdipoR1 (C) and R2 (D) mRNAs in skeletal muscles of control mice and mice treated with STZ without or with insulin. Preparation of total RNA and quantification of AdipoRs mRNAs were done as described in FIG. 1. Each bar represents the mean±s.e. (n=3). (*, P<0.05; **, P<0.01; compared with STZ mice without insulin).

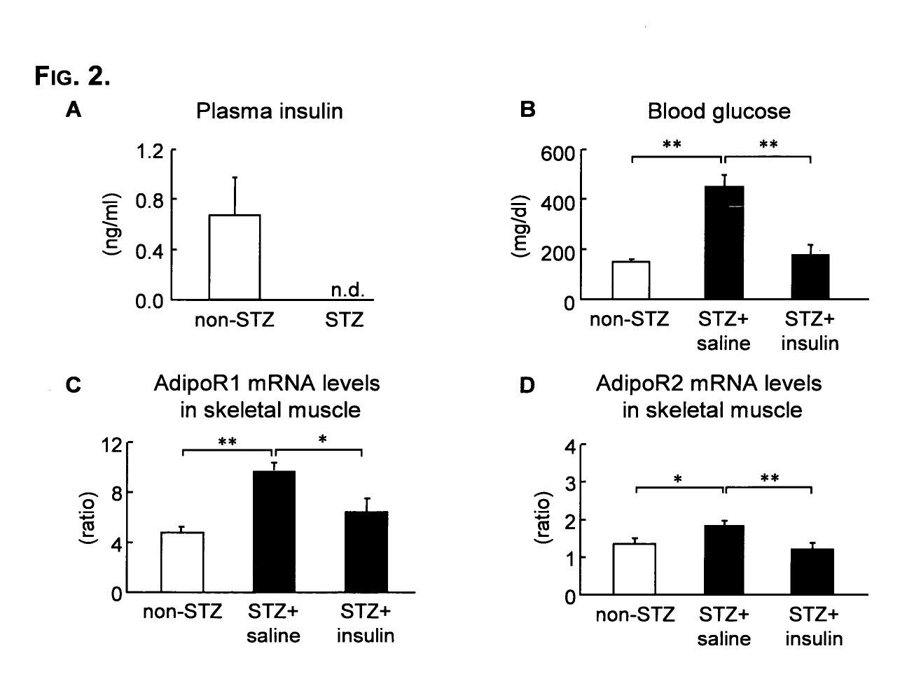

FIG. 3. Amounts of mRNAs for AdipoR1 (A, C, E) and AdipoR2 (B. D, F) in mice hepatocytes (A, B ) or C2C12 myocytes (C-F) treated with or without insulin incubated with or without LY 294002 or PD98059 (C, D) transfected with an adenovirus containing LacZ or Foxo1. Hepatocytes were isolated from mice livers and incubated as described in Experimental prodedures. The cell monolayers were then incubated for 6 h with or without indicated concentration of insulin. Preparation of total RNA and quantification of AdipoRs mRNAs were done as described in FIG. 1. Each bar represents the mean±s.e. (n=3). (*, P<0.05; **, P<0.01; compared with cells treated with vehicle).

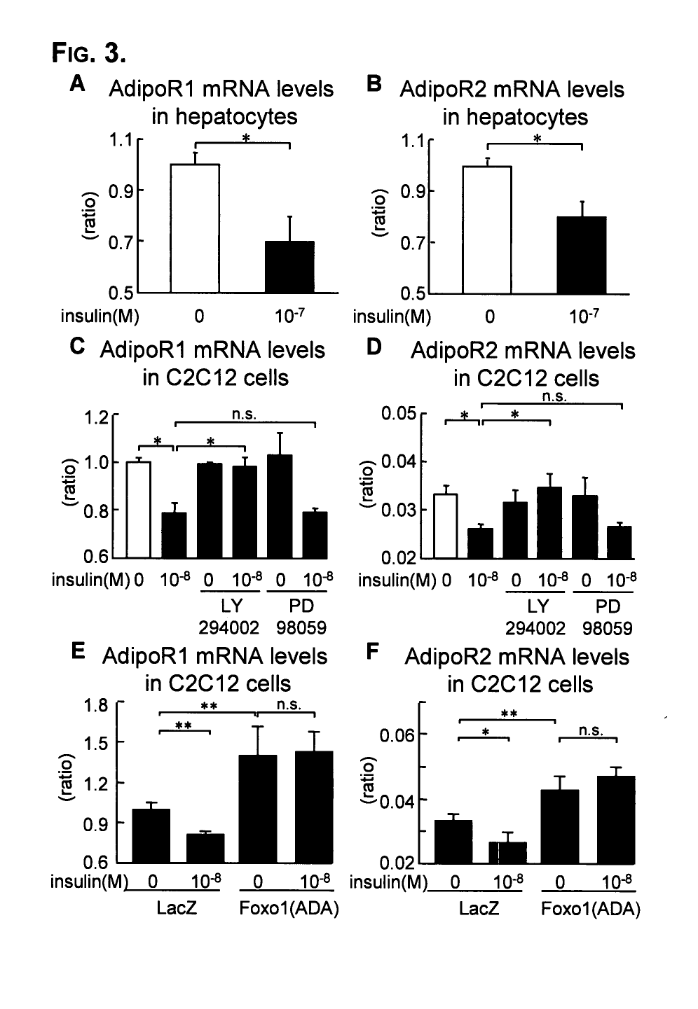

FIG. 4. Expressions of AdipoR1 (A) and AdipoR2 (B) mRNAs in liver, soleus, EDL, WAT and BAT, blood glucose (C) and plasma insulin (D) of wild-type and ob/ob mice. Preparation of total RNA and quantification of AdipoRs mRNAs were done as described in FIG. 1. Each bar represents the mean±s.e. (n=3). (*, P<0.05; **, P<0.01; between wild-type and ob/ob mice).

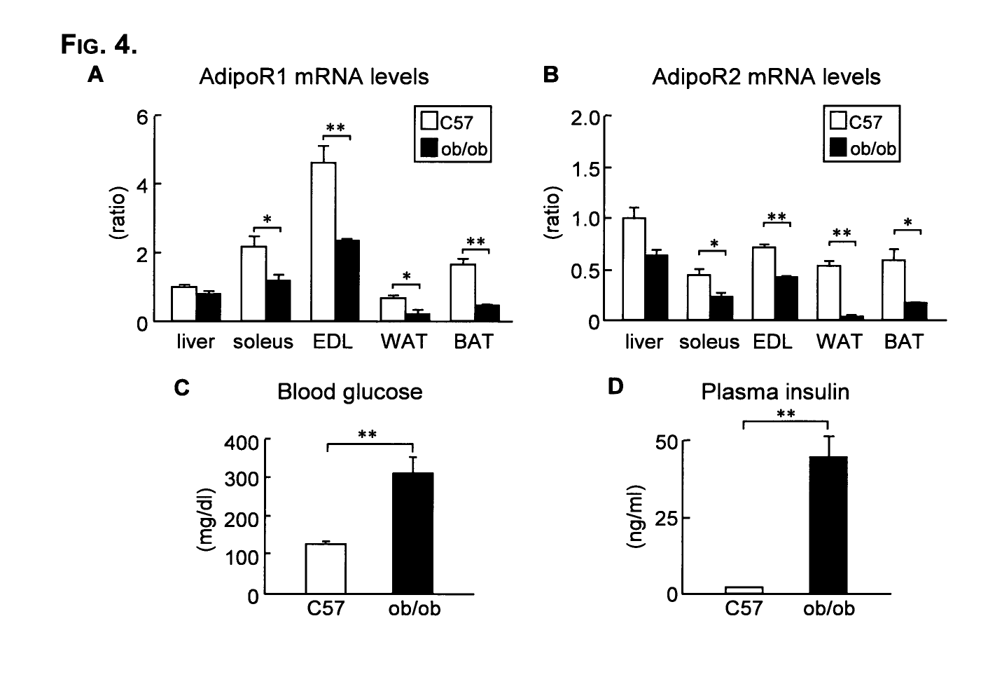

FIG. 5. Binding affinity of globular (gAd) (A) or full-length adiponectin (Ad) (B) to the membrane fractions of skeletal muscle. Phosphorylation of the a subunit of AMPK (C) in skeletal muscles from wild-type (C57) or ob/ob mice treated with the indicated concentrations (50 μg/10 g body weight) of full-length adiponectin (Ad) for 5 min. Immunoblot analyses were performed using anti-p-AMPK and anti-AMPK. Each bar represents the mean±s.e. (n=5-7). (*, P<0.05; **, P<0.01; compared with C57).

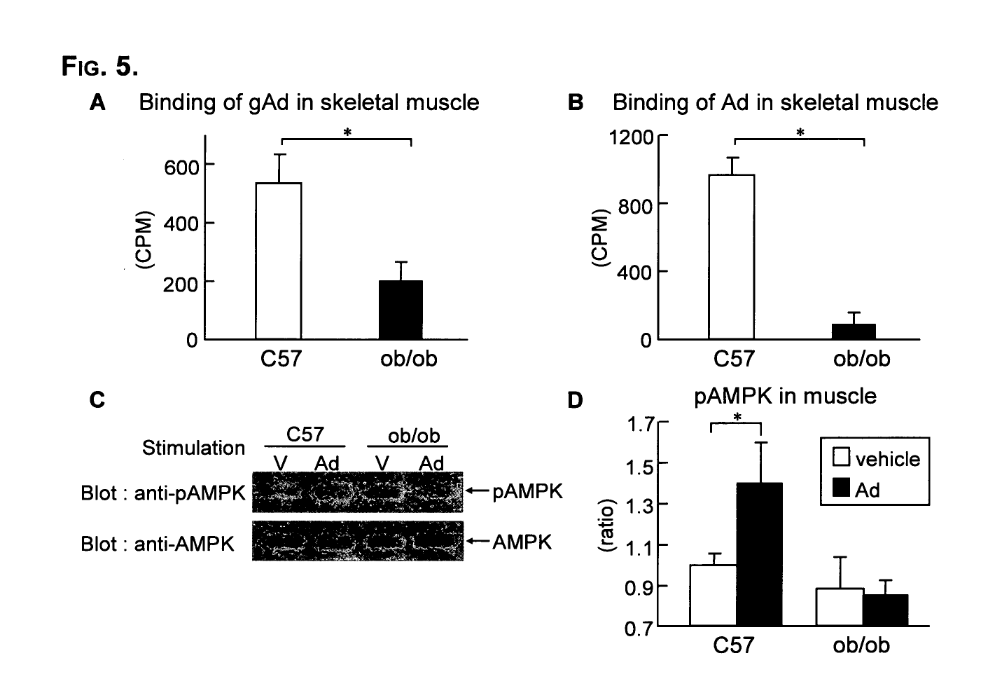

1-17. (canceled)

18. A method comprising: treating cells with an agent that inhibits insulin-induced suppression of adiponectin receptor expression under conditions such that said agent inhibits insulin-induced suppression of adiponectin receptor expression by said cells.

19. The method of claim 18, wherein said agent comprises a FoxoI gene.

20. The method of claim 18, wherein said agent comprises a FoxoI protein.

21. The method of claim 18, wherein said agent is a PI3-kinase pathway inhibitor.

22. The method of claim 19, wherein said PI3-kinase pathway inhibitor comprises LY294002.

23. The method of claim 18, wherein said adiponectin receptor comprises adiponectin receptor 1.

24. The method of claim 18, wherein said adiponectin receptor comprises adiponectin receptor 2.

25. The method of claim 18, wherein said cells comprise hepatocytes.

26. The method of claim 18, wherein said cells comprise myocytes.

27. The method of claim 18, wherein said cells are in a mammalian subject.

28. The method of claim 27, wherein said mammalian subject has one or more conditions selected from the group consisting of adiponectin resistance, insulin resistance, obesity, type II diabetes and arteriosclerosis.

29. A method of screening a test compound, comprising:
a) treating cells expressing an adiponectin receptor with a test compound in the presence and absence of insulin to produce treated cells;
b) measuring adiponectin receptor expression by said treated cells; and
c) identifying said test compound as inhibiting insulin-induced suppression of adiponectin receptor expression when said adiponectin receptor expression by said cells is reduced to a lesser amount in the presence of said test compound than in its absence.

30. The method of claim 29, wherein said adiponectin receptor comprises adiponectin receptor 1.

31. The method of claim 29, wherein said adiponectin receptor comprises adiponectin receptor 2.

32. The method of claim 29, wherein said cells comprise hepatocytes.

33. The method of claim 29, wherein said cells comprise myocytes.

34. A method of screening a test compound, comprising:
a) contacting PI3-kinase with a PI3-kinase substrate in the presence and absence of a test compound;
b) measuring PI3 kinase activity; and
c) identifying said test compound as inhibiting insulin-induced suppression of adiponectin receptor expression when said PI3 kinase activity is reduced in the presence of said test compound.

35. A method of screening a test compound, comprising:
a) contacting Akt with a FoxoI in the presence and absence of said test compound;
b) measuring phosphorylation of FoxoI; and
c) identifying said test compound as inhibiting insulin-induced suppression of adiponectin receptor expression when phosphorylation of said FoxoI is reduced in the presence of said test compound.

36. A method for suppressing adiponectin receptor expression comprising: treating cells with insulin or insulin gene under conditions such that said insulin or said insulin gene suppresses adiponectin receptor expression by said cells.
